(19) United States
(12) Patent Application Publication
(10) Pub. No.: US 2008/0160000 A1
(43) Pub. Date: Jul. 3, 2008

(54) COCCIDIOSIS AND CLOSTRIDIAL DISEASE PROPHYLACTIC AND/OR THERAPEUTIC FEED FOR COCCIDIOSIS AND CLOSTRIDIAL DISEASE

(75) Inventors: Yukihiro Motozono, Chuo-ku (JP); Hiroyuki Suzuki, Nasushiobara-shi (JP); Kazuhiro Hatano, Nasushiobara-shi (JP); Masaaki Kise, Nasushiobara-shi (JP); Shinichi Horigome, Higashimurayama-shi (JP); Katsuhiro Mori, Kodaira-shi (JP); Yoshinaga Doi, Bunkyo-ku (JP); Ryoji Koga, Zama-shi (JP)

Correspondence Address: OBLON, SPIVAK, MCCLELLAND MAIER & NEUSTADT, P.C., 1940 DUKE STREET, ALEXANDRIA, VA, 22314, US

(73) Assignee: Marubeni Nisshin Feed Co., Ltd., Chuo-ku (JP)

(21) Appl. No.: 11/923,985

(22) Filed: Oct. 25, 2007

(30) Foreign Application Priority Data
Oct. 31, 2006 (JP) 2006-295872

(51) Int. Cl. A61K 35/74; A61K 31/05; A61K 36/06; A61K 36/752

(52) U.S. Cl. 424/93.45; 514/733; 424/736; 424/93.51

(57) ABSTRACT

There is provided an animal feed, an anticoccidial agent, and an anticlostridial agent having less harm and an excellent prophylactic and/or therapeutic effect for coccidiosis and clostridial disease. Also provided is a prophylactic and/or therapeutic feed for coccidiosis containing pinene, thymol, eugenol, and limonene; an anticoccidial agent containing pinene, thymol, eugenol, and limonene as active ingredients; a clostridial disease prophylactic and/or therapeutic feed containing pinene, thymol, eugenol, and limonene; and an anticlostridial agent containing pinene, thymol, eugenol, and limonene as active ingredients.

### 1. FIELD OF THE INVENTION

The present invention relates to a prophylactic and/or therapeutic feed and an anticoccidial agent for coccidiosis of an animal. The present invention also relates to a prophylactic and/or therapeutic feed and an anticlostridial agent for clostridial disease of an animal.

### 2. DESCRIPTION OF THE RELATED ART

Coccidiosis of poultry such as chickens, turkey, ducks, quail, geese, and pheasants or livestock such as rabbits, cattle, sheep, and pigs is a contagious disease caused by infection with some parasitic protozoa. Coccidiosis has spread worldwide. In the case of a chicken, it is known that coccidiosis is caused by parasitic protozoa such as Eimeria tenella, Eimeria acervulina, Eimeria necatrix, Eimeria mitis, Eimeria maxima, Eimeria praecox, Eimeria hagani, and Eimeria brunette.

In the case of a turkey, it is known that coccidiosis is caused from parasitic protozoa such as Eimeria meleagridis, Eimeria dispersa, Eimeria gallopavonis, Eimeria innocua, and Eimeria adenoeides.

In the case of ducks, it is known that coccidiosis is caused from parasitic protozoa such as Tyzzeria perniciosa.

In the case of geese, it is known that coccidiosis is caused by parasitic protozoa such as Eimeria truncata, Eimeria anseris, Eimeria nocens, and Eimeria parvula.

In the case of pheasants, it is known that coccidiosis is caused by parasitic protozoa such as Eimeria phasiani and Eimeria dispersa.

In the case of cattle, it is known that coccidiosis is caused by parasitic protozoa such as Eimeria zurni, Eimeria bovis, and Eimeria ellipsoidalis.

An animal infected with the parasitic protozoa shows symptom such as diarrhea, or hematochezia. If treatment is delayed or if the symptom is serious, the animal may die. Although the animal does not die, enormous damage to productivity occurs. Therefore, coccidiosis is one of the serious animal diseases. Particularly, coccidiosis becomes a problem to animal breeders who breed a large herd of poultry.

Conventionally, in order to prevent or treat coccidiosis, an antibiotic, a chemical therapeutic agent made of synthetic antibacterials, and a biological agent such as vaccine have been mainly used.

However, the antibiotic or the chemical therapeutic agent has a problem of occurrence of a side effect and deterioration in efficacy due to resistance to the agent. The biological agent has a problem that the agent cannot be used for treatment but prevention. If a person eats meat, egg or the like of an animal, to which the agents are administered, the residual agents in the body of the animal or the like are transferred to the human body. Therefore, an amount of the administered agents and an administration time interval are very limited.

As an example of a coccidiosis prophylactic and/or therapeutic agent having less harm without the aforementioned problems, a coccidiosis mitigative agent containing cashew nut shell liquid and/or anacardic acid as active ingredients is proposed (JP-A No. 8-231410). In addition, a feed additive containing green tea and/or a green tea extract solution capable of obtaining an anticoccidial effect is disclosed (JP-A No. 7-255386).

However, a sufficiently satisfactory effect cannot be obtained by using the conventional feeds.

Clostridium perfringens infection of livestock and poultry is a disease caused by infection with Clostridium perfringens. As a representative symptom thereof, there is gangrenous enteritis. In addition, clostridium perfringens infection causes enterotoxemia and malignant edema. Since morbidity and lethality are high, poultry farmers, broiler farmers, pig breeders, and dairy farmers suffer very heavy economic damage. Therefore, clostridium perfringens infection is one of the most serious diseases that need to be rapidly prevented and eradicated among various diseases of livestock and poultry.

Gangrenous enteritis in baby pigs or chicks has a clinical pathological characteristic that diarrhea occurs and infiltration of bacilli into the intestinal mucousa, and necrosis of the small intestine are involved.

Enterotoxemia is a disease caused by proliferation of Clostridium perfringens in the small intestine of an animal. Due to the proliferation, Clostridium perfringens generates a toxin, so that necrotic or hemorrhage lesion occurs. Furthermore, due to the involved toxemia, the infected animal may undergo an acute death.

Malignant edema is a disease caused by infiltration of bacilli into a wounded surface formed accidentally or during a surgical operation. The infiltrated bacilli germinate and proliferate to generate a toxin, so that the infected animal undergoes death from the involved toxemia and bacillemia.

As an example of the prophylactic and/or therapeutic agent for clostridial disease, there is disclosed clostridium perfringens prophylactic and therapeutic agent for livestock and poultry, which contains one or two or more natural medicines selected from the group consisting of licorice, artemisia, aureum, axseed, cork tree bark, geranium thunbergii, Magnolia obovata Thunb, salvia militiorhhiza, anemarrhenae rhizoma, pieplant, clove, ligustrum pericei hayata, schizonepetae spica, cinnamon, scrophularia buergerianamiq, c. occidentalis, eriobotryae folium, parsnip, hop, perillae herba, myricae cortex, forsythia suspensa, aloe, oxtongue, clematidis radix, prunus mume, plectranthus japonicus, plantago, magnoliae flos, artemisiae capillari flos, aloeswood, cnidii rhizoma, ligusticum sinense, gobayashi, Japanese cornel, lithospermi radix, picrorhiza kurrooa, peony root, rosa laevigata michx, thyme, white nandin, sanguisorbae radix, and ephedra (Japanese Patent No. 2599161).

However, although clostridium perfringens infection is a very serious contagious disease, prevention and treatment methods are not yet established. Although proper breeding management has been carried out, prevention is very difficult in fact.

In consideration of the aforementioned conventional problems, an object of the present invention is to provide an animal feed, anticoccidial agent, and anticlostridial agent having less harm and an excellent preventive and/or therapeutic effect for coccidiosis and clostridial disease.

### SUMMARY OF THE INVENTION

The inventors have researched so as to solve the aforementioned problems. As a result, the inventors found out that an excellent prophylactic and/or therapeutic effect for coccidiosis and clostridial disease can be obtained by feeding four ingredients of pinene, thymol, eugenol, and limonene or a natural substance containing the ingredients to an animal, so that the invention was contrived.

In order to solve the problems, the present invention provides a prophylactic and/or therapeutic feed for coccidiosis containing pinene, thymol, eugenol, and limonene.

In order to solve the problems, the present invention provides a prophylactic and/or therapeutic feed for coccidiosis containing nutmeg, thyme, clove, and citrus.

In order to solve the problems, the present invention provides an anticoccidial agent containing pinene, thymol, eugenol, and limonene as active ingredients.

In order to solve the problems, the present invention provides an anticoccidial agent containing nutmeg, thyme, clove, and citrus.

In order to solve the problems, the present invention provides a prophylactic and/or therapeutic feed for clostridial disease containing pinene, thymol, eugenol, and limonene.

In order to solve the problems, the present invention provides a prophylactic and/or therapeutic feed for clostridial disease containing nutmeg, thyme, clove, and citrus.

In order to solve the problems, the present invention provides an anticlostridial agent containing pinene, thymol, eugenol, and limonene as active ingredients.

In order to solve the problems, the present invention provides an anticlostridial agent containing nutmeg, thyme, clove, and citrus.

When a feed or an agent according to the present invention is fed or administered to an animal, coccidiosis and clostridial disease of the animal can be prevented and/or treated without an occurrence of a side effect. A decrease in breeding yield and a deterioration in productivity caused by coccidiosis or clostridial disease can be prevented.

### DESCRIPTION OF THE PREFERRED EMBODIMENT

Pinene, thymol, eugenol, and limonene used in the present invention may be a chemical synthetic substance or any substance separated from a natural substance. A bulk of a natural substance containing pinene, thymol, eugenol, and limonene may be directly used. Alternatively, a concentrated substance of active ingredients obtained by distilling or extracting the natural substance may be used. In the case of the natural substance, the concentrated substance of the active ingredients is preferably used in terms of an anticoccidial effect and anticlostridial effect. Particularly, a refined substance thereof is more preferable.

Examples of a natural substance containing pinene include nutmeg, coriander, and lemon, and particularly, nutmeg (Myristica fragrans) is preferable. An example of a natural substance containing thymol includes thyme, and particularly, thyme (Thymus vulgaris) is preferable. Examples of a natural substance containing eugenol include cinnamon and clove, and particularly, clove (Syzygium aromaticum Eugenia caryophyllata) is preferable. Examples of a natural substance containing limonene include citrus, peppermint, spearmint and fennel, and particularly, citrus (Citrus spp.) is preferable. Examples of the citrus include lemon, lime, orange, and bigarade.

In addition, pinene has some kinds of types such as α-pinene and β-pinene in nature. In the present invention, the aforementioned compounds may used either alone or mixture thereof.

An extract is directly extracted from the natural substance containing pinene, thymol, eugenol, and limonene by using an organic solvent. Alternatively, after a pre-process such as drying, cutting, grinding, powdering, and the like is performed on the natural substance as needed, an extract may be extracted from the natural substance by using an organic solvent. For the extraction, any portions of the natural substance can be used. Preferably, a nucleolus in a seed nucleus of a fruit of nutmeg, a leaf or a flower spike of thyme, a bud of clove, and a pericarp of citrus are used.

As an extraction method, a general method, for example, a solvent extraction method may be used.

Examples of the organic solvent used for the solvent extraction method include an alcohol such as methanol, ethanol, propanol, and butanol; a ketone such as acetone and methyl ethyl ketone; an ester such as methyl acetate and ethyl acetate; a chain or cyclic ether such as tetrahydrofuran and diethyl ether; a halogenated hydrocarbon such as dichloromethane, chloroform, and carbon tetrachloride; a hydrocarbon such as hexane, cyclohexane, petroleum ether; an aromatic hydrocarbon such as benzene and toluene; a pyridine; a supercritical carbon dioxide; a fatty oil, a wax, and other oils. The solvent may be used either alone or two or more thereof. A replacement of the solvents may be repeatedly performed.

In addition, a roughly refined substance obtained by performing hydrodistillation on a nucleolus in a seed nucleus of a fruit of nutmeg, a leaf or a flower spike of thyme, a bud of clove, or a pericarp of citrus may be used.

Pinene, thymol, eugenol, and limonene that can be obtained by synthesis or separation from the natural substance can be used in any type of a compound, an extract, a roughly refined substance, or a refined substance with a proviso that the product is allowable in regulations for medicine, food, and feed. Examples of a refining mean include distillation, organic solvent sedimentation, centrifugal separation, critical membrane filter, a high performance liquid chromatography, and a column chromatography.

As shown in the later-described example, the four ingredients of pinene, thymol, eugenol, and limonene or the natural substance containing the ingredients are used in combination thereof, and an excellent prophylactic and/or therapeutic effect for coccidiosis and clostridial disease of an animal can be obtained. In addition, by adding the four ingredients or the natural substance containing the four ingredients to a feed, a prophylactic and/or therapeutic effect for coccidiosis disease and clostridial disease of various animals can be obtained. In addition, by adding and mixing the four ingredients or the natural substance containing the four ingredients thereto, an anticoccidial agent and an anticlostridial agent can be produced. Such agents may be directly administered to an animal, or a feed added with the agents may be fed to an animal.

In the present invention, amounts of pinene, thymol, eugenol, and limonene contained in the feed are different according to a type of animals, years of age (or months of age), symptoms, or the like. In general, the adding amount are preferably in a range of 0.0001 to 0.1 mass % (external division) of the feed to which the four ingredients are not yet added, and more preferably, in a range of 0.0005 to 0.01 mass %. If the adding amount is less than 0.0001 mass %, it is difficult to obtain effects for coccidiosis and clostridial disease. On the other hand, if the adding amount is more than 0.1 mass %, costs disadvantageously increase. In addition, a mixture ratio of pinene, thymol, eugenol, and limonene is not particularly limited but can be suitably selected. Preferably, the mixture ratio is in range of (1 to 100):(1 to 100):(1 to 100):(1 to 100) in terms of an anticoccidial effect, an anticlostridial effect, and a preference to an animal.

In addition, the amount of the natural substance containing pinene, thymol, eugenol, and limonene such as nutmeg, thyme, clove, and citrus added to a feed is preferably in a range of 0.01 to 10 mass % (external division) in an equivalent dry matter, and more preferably, in a range of 0.05 to 5 mass %.

A raw material for a feed according to the present invention is not limited to a specific one. Examples thereof include a grain such as a corn, milo, barley, and wheat; chaff and bran such as wheat bran; vegetable oilcake such as soybean oilcake and rapeseed cakeoil; animal by-product feed such as fish meal and bone meal; minerals such as a dietary salt, oligosaccharide, hydrated silica, various vitamins, calcium carbonate, calcium phosphate; amino acids; and organic acids.

In the present invention, amounts of pinene, thymol, eugenol, and limonene contained in the anticoccidial agent or the anticlostridial agent are different according to a type of animals, years of age (or months of age), symptoms, or the like. The amounts are preferably in a range of 0.01 to 60 mass % in the agent, and more preferably, in a range of 0.1 to 10 mass %. If the adding amount is less than 0.01 mass %, the administered amount of the anticoccidial agent or the anticlostridial agent needs to be large, so that it is difficult to administer the agent in a short time. On the other hand, if the adding amount is more than 60 masse, it is difficult to produce and treat the agent. In addition, a mixture ratio of pinene, thymol, eugenol, and limonene is not particularly limited but can be suitably selected. Preferably, the mixture ratio is in range of (1 to 100):(1 to 100):(1 to 100):(1 to 100) in terms of an anticoccidial effect, an anticlostridial effect, and a preference to an animal.

In addition, in a case where the natural substance containing pinene, thymol, eugenol, and limonene such as nutmeg, thyme, clove, or citrus is added, the mixture ratio is preferably in a range of 1 to 100 mass % in equivalent dry matter, and more preferably, in a range of 10 to 50 mass %.

The agent according to the present invention is preferably produced as a pill, a capsule, a granule, a powder, or a syrup by using a pharmaceutically acceptable container, for example, an excipient, a lubricant, a diluent, a coupling agent, a destructive agent, an emulsifying agent, a stabilizing agent, a corrective agent, and the like. In addition, as described above, a feed added with the agent according to the present invention is preferably fed to an animal.

Furthermore, a mixture of the feed or the agent according to the present invention with Saccharomyces and Lactobacillus can improve a prophylactic and/or therapeutic effect for coccidiosis and clostridial disease.

Examples of Saccharomyces used in the present invention, include Saccharomyces such as Saccharomyces cerevisiae, Saccharomyces uvarum, Saccharomyces diastaticus, and Saccharomyces rouxii; Schizosaccharomyces such as Schizosaccharomyces pombe; Saccharomycodes such as Saccharomycodes ludwigii; Hanseniaspora such as Hanseniaspora valbyensis; Kluyveromyces such as Kluyveromyces fragilis and Kluyveromyces lactis; Pichia such as Pichia membranaefaciens; Hansenula such as Hansenula anomala; Debaryomyces such as Debaryomyces hansenii; Lipomyces such as Lipomyces starkeyi; Brettanomyces such as Brettanomyces bruxellensis; and Candida such as Candida utilis, Candida tropicalis, and Candida lipolytica.

In addition to the aforementioned Saccharomyces, a cell wall of Saccharomyces separated from the cell of Saccharomyces by using a general method can be also used for Saccharomyces according to the present invention. In general, the cell wall of Saccharomyces contains Saccharomyces and/or a product of zymolysis using saccharomyces. One or two or more of the aforementioned Saccharomyces can be suitably selected.

Examples of Lactobacillus according to the present invention include Lactobacillus acidophilus and Enterococcus faecium. One or two or more of the aforementioned Lactobacillus can be suitably selected. In general, an amount of Lactobacillus contained in the product is in a range of 10⁵/g to 10¹²/g.

In the present invention, amounts of the Saccharomyces and the Lactobacillus contained in the feed are as follows, in terms of an anticoccidial effect, anticlostridial effect, and a preference to an animal. The amount of Saccharomyces is preferably in a range of 0.01 to 5 mass %, and more preferably, in a range of 0.05 to 0.5 mass %. The amount of the Lactobacillus is preferably in a range of 0.001 to 10 mass %, and more preferably, in a range of 0.001 to 1 mass %.

In the present invention, amounts of Saccharomyces and Lactobacillus contained in the anticoccidial agent or the anticlostridial agent are as follows, in terms of an anticoccidial effect, anticlostridial effect, and a preference to an animal. The amount of Saccharomyces is preferably in a range of 1 to 99.9 mass %, and more preferably, in a range of 5 to 50 mass %. The amount of Lactobacillus is preferably in a range of 0.01 to 50 mass %, and more preferably, in a range of 0.1 to 20 mass %.

In the present invention, a feeding amount of the feed or an administering amount of the agent is different according to a type of animals, years of age (or months of age), symptoms, or the like. In general, an amount of pinene, thymol, eugenol, and limonene contained therein are preferably in a range of 0.05 to 200 mg/body weight kg/day, and more preferably, in a range of 0.2 to 20 mg/body weight kg/day. In the present invention, the feeding of a feed or the administering of an agent may be carried out over the whole breeding time interval or a predetermined portion of the breeding time interval. In particularly, in the case of chickens, feeding is preferably carried out for four weeks from the birth thereof so as to obtain a high anticoccidial or anticlostridial effect.

In the present invention, the animal which the feed or the agent is fed to is not particularly limited to a specific one. Poultry such chickens and ducks and livestock such as cattle, pigs, and sheep may be exemplified, and chickens are particularly preferred.

### EXAMPLES

Hereinafter, exemplary examples of the present invention will be descried in detail, but the present invention is not limited thereto.

At first, chicken feeds (basal feeds) having a composition shown in the following Table 1 are prepared.

TABLE 1

| | | | Example 3 | | | |
|---|---|---|---|---|---|---|
| | | | 0 to 2 | 2 to 4 | 4 to 5 | |
| | Example 1 | Example 2 | weeks | weeks | weeks | Example 4 |
| Corn (containing 12% | 69.34 | 65.47 | 65.18 | 65.74 | 73.17 | 34.23 |
| soybean meal) | | | | | | |
| Rye | | | | | | 30.00 |
| Soybean meal | 12.30 | 18.44 | 20.28 | 17.31 | 12.72 | 18.89 |
| Rapeseed meal | | 2.00 | 2.00 | 2.00 | 2.60 | |
| Gluten meal | | 3.00 | 2.00 | 3.00 | | 3.00 |
| Fish meal | 15.00 | 5.00 | 5.00 | 5.00 | 4.00 | 7.00 |
| Animal fat | 1.70 | 3.40 | 2.80 | 4.20 | 5.20 | 4.38 |
| Dietary salt | 0.23 | 0.35 | 0.35 | 0.34 | 0.24 | 0.26 |
| Calcium carbonate | 1.06 | 1.44 | 1.45 | 1.44 | 1.34 | 1.02 |
| Tribasic calcium | | 0.48 | 0.46 | 0.50 | 0.43 | 0.80 |
| phosphate | | | | | | |
| Methionine | 0.11 | 0.12 | 0.16 | 0.13 | 0.15 | 0.17 |
| Lysine | | 0.04 | 0.06 | 0.08 | | |
| Vitamin mineral mix | 0.20 | 0.18 | 0.18 | 0.18 | 0.09 | 0.18 |
| Choline chloride 60% | 0.06 | 0.06 | 0.06 | 0.06 | 0.04 | 0.07 |
| Phytase | | 0.02 | 0.02 | 0.02 | 0.02 | |
| Total | 100.00 | 100.00 | 100.00 | 100.00 | 100.00 | 100.00 |
| (Nutrient) | | | | | | |
| CP (%) | 23.0 | 22.3 | 22.3 | 21.9 | 18.3 | 22.3 |
| ME (Kcal/Kg) | 3100 | 3103 | 3054 | 3153 | 3230 | 3052 |
| Ca (%) | 1.0 | 1.0 | 1.0 | 1.0 | 0.9 | 1.0 |
| P (%) | 0.6 | 0.6 | 0.6 | 0.6 | 0.5 | 0.7 |

### Example 1

Seven groups (30 chickens/group) of test chickens (type of chickens: chunky, one week of age, male) were prepared.

The test chickens of the first to seventh groups are bred for 12 days by freely feeding the feeds (Table 4) obtained by adding the ingredients to the basal feed (100 mass %) shown in Table 1. The test chickens of the second to seventh groups excluding the first group were infected with oocysts of coccidial protozoa Eimeria acervulina in a ratio of 5.0×10⁴ oocysts/chicken in an oral administration manner on the seventh day after the start of the breeding. Next, after the breeding was finished, the test chickens of the groups were dissected, and degrees of lesions in the duodenums were evaluated according to the following evaluation criteria. In addition, on the dissection day, feces of the groups were sampled, and the number of oocysts per gram of the feces (OPG) was measured. OPG scores were determined by OPG values according to the evaluation criteria as follow. The results are shown in Table 5.

TABLE 2
Lesion in duodenum

| | Evaluation | |
|---|---|---|
| Symbols | Scores | Descriptions |
| − | 0 point | Any lesion is not detected |
| + | 1 point | Amorphous white necrosis occurs in several |
| | | portions |
| ++ | 2 points | Necrotic portions are distributed in the duodenum |
| +++ | 3 points | A large number of necrotic portions occur in the |
| | | duodenum, and intestinal tract are swollen and its |
| | | contents are watery. |
| ++++ | 4 points | Necrotic portions occur in the entire of the |
| | | duodenum, and intestinal contents are creamy. |

TABLE 3
OPG score in feces

| Estimation | |
|---|---|
| Scores | OPG Values |
| 0 point | Equal to or more than 0 and less than 1.0 × 10⁵ |
| 1 point | Equal to or more than 1.0 × 10⁵ and less than 1.0 × 10⁶ |
| 10 points | Equal to or more than 1.0 × 10⁶ and less than 5.0 × 10⁶ |
| 20 points | Equal to or more than 5.0 × 10⁶ and less than 1.0 × 10⁷ |
| 40 points | Equal to or more than 1.0 × 10⁷ |

TABLE 4

| Test Group | |
|---|---|
| Nos. | Contents of Feeds |
| First Group | Basal feed (non-infected) |
| Second Group | Basal feed (infected) |
| Third Group | Basal feed + Salinomycine 50 ppm |
| Fourth Group | Basal feed + Mixture 90 ppm containing thymol (3%), |
| | eugenol (1%), and limonene (2%) |
| Fifth Group | Basal feed + Mixture 90 ppm containing pinene (4%), |
| | thymol (3%), eugenol (1%), and limonene (2%) |
| Sixth Group | Basal feed + Mixture 90 ppm containing thymol (3%), |
| | eugenol (1%), limonene (2%) + cumin aldehyde (4%) |
| Seventh Group | Basal feed + Mixture 450 ppm containing pinene (0.2%), |
| | limonene (6%), cineol (5%), and β-phellandrene (1%) |

* Salinomycine: Manufactured by Kaken Pharmaceutical Co., Ltd.; thymol, eugenol, limonene, pinene, cumin aldehyde, cineol, and β-phellandrene: Manufactured by TAKASAGO INTERNATIONAL CORPORATION

TABLE 5

| | | First | Second | Third | Fourth | Fifth | Sixth | Seventh |
|---|---|---|---|---|---|---|---|---|
| Tested Group Nos. | | group | group | group | group | group | group | group |
| Weights | Average initial body | 419 | 420 | 402 | 421 | 416 | 417 | 413 |
| | weight (g) | | | | | | | |
| | Average final body | 684 | 628 | 643 | 622 | 655 | 629 | 629 |
| | weight (g) | | | | | | | |
| | Body weight gain | 266 | 208 | 242 | 201 | 239 | 212 | 215 |
| | (g/chicken) | | | | | | | |
| | Feed intake | 411 | 274 | 318 | 295 | 293 | 290 | 297 |
| | (g/chicken) | | | | | | | |
| | Feed conversion ratio | 1.55 | 1.32 | 1.32 | 1.47 | 1.23 | 1.37 | 1.38 |
| | Weight gain/initial | 63.4 | 49.4 | 60.1 | 47.8 | 57.4 | 50.7 | 52.0 |
| | weight ratio (%) | | | | | | | |
| | Relative ratio weight | 100.0 | 78.0 | 95.0 | 75.0 | 90.0 | 80.0 | 82.0 |
| | gain/initial weight | | | | | | | |
| | (compared with First | | | | | | | |
| | group) (%) A | | | | | | | |
| Infection | Survival ratio (%) B | 100.0 | 100.0 | 100.0 | 100.0 | 97.0 | 100.0 | 97.0 |
| indices | Average lesion score | 0.0 | 3.9 | 3.6 | 3.9 | 3.7 | 3.8 | 3.9 |
| | C | | | | | | | |
| | Average OPG score in | 0.0 | 40.0 | 33.3 | 33.3 | 33.3 | 40.0 | 33.3 |
| | feces D | | | | | | | |
| | ACI (anticoccidial | 200 | 99 | 126 | 103 | 116 | 102 | 106 |
| | indces) (*1) | | | | | | | |

* D: Average OPG scores in feces: Each average OPG score in feces is an average value of three tested bodies in each group.
(*1) ACI = (A + B) − (10C + D)

Referring to the results shown in Table 5, when the feed added with pinene, thymol, eugenol, and limonene was fed (to the fifth group), the retardation in growth caused by infection with Eimeria acervulina observed in the second group fed with the basal feed (infected) can be alleviated. In addition, lesion indices of duodenums can be suppressed down to a lower level. An anticoccidial index (ACI) estimated from a weight increment ratio, a survival ratio, a lesion score, and OPG score in feces was the highest next to that of salinomycine. As a result, it was determined that the influence of Eimeria acervulina to the productivity could be suppressed down to a low level by the composition of pinene, thymol, eugenol, and limonene.

### Example 2

Three groups (20 chickens/group) of tested chickens (type of chickens: chunky, neonatal chick, male) were prepared.

The test chickens of the eighth to tenth groups are bred for 28 days by freely feeding the feeds (Table 6) obtained by adding the ingredients to the basal feed (100 mass %) shown in Table 1. The test chickens of all the groups were infected with oocysts of coccidial protozoa Eimeria maxima in a ratio of 1.5×10⁴ oocysts/chicken in an oral administration manner on the twenty-first day after the breeding. On each sixth to eighth day after the infection, the feces of the groups were sampled, and the number of oocysts per gram of the feces (OPG) was measured (as a three-day average value). The results are shown in Table 7.

TABLE 6

| Test Group Nos. | Contents of Feeds |
|---|---|
| Eighth Group | Basal feed |
| Ninth Group | Basal feed + salinomycine 50 ppm |
| Tenth Group | Basal feed + Mixture 90 ppm containing pinene (4%), |
| | thymol (3%), eugenol (1%), and limonene (2%) |

* Salinomycine: Manufactured by Kaken Pharmaceutical Co., Ltd.; pinene, thymol, eugenol, and limonene: Manufactured by TAKASAGO INTERNATIONAL CORPORATION

TABLE 7

| ( ) Compared-Group | | Eighth | Ninth | |
|---|---|---|---|---|
| Comparison | | Group | Group | Tenth Group |
| Initial body | (g/chicken) | 36(100) | 36(100) | 36(100) |
| weight | | | | |
| Final body weight | (g/chicken) | 904(100) | 1054(117) | 994(110) |
| Body weight gain | (g/chicken) | 868(100) | 1018(117) | 959(110) |
| Feed intake | (g/chicken) | 1383(100) | 1525(110) | 1487(108) |
| Feed conversion | | 1.60(100) | 1.50(94) | 1.55(97) |
| ratio | | | | |
| Growth ratio | (%) | 100(100) | 100(100) | 100(100) |
| Maxima OPG (*) | (10,000 | 17.8(100) | 3.1(18) | 11.4(64) |
| | oocysts/g | | | |
| | of feces) | | | |

(*) Maxima OPG: Average on the 6 to 8 days after infection

Referring to the results shown in Table 7, when the feed added with pinene, thymol, eugenol, and limonene was fed (to the tenth group), the excretion of Eimeria maxima OPG was reduced, so that the growth in the infected state of Eimeria maxima can be improved. As a result, it was determined that the influence of the Eimeria maxima to the productivity could be suppressed down to a low level by the composition of pinene, thymol, eugenol, and limonene.

### Example 3

Five groups (80 chickens/group) of test chickens (type of chickens: chunky, neonatal chick, male) were prepared.

The tested chickens of the eleventh to fifteenth groups are bred for 35 days by freely feeding the feeds (Table 8) obtained by adding the ingredients to the basal feed (100 mass %) shown in Table 1 while changing the mixture composition of the basal feed every two weeks. The test chickens of all the groups were infected with oocysts of coccidial protozoa Eimeria acervulina in a ratio of 7.0×10³ oocysts/chicken, oocysts of coccidial protozoa Eimeria tenella in a ratio of 6.0×10³ oocysts/chicken, and oocysts of coccidial protozoa Eimeria maxima in a ratio of 1.5×10⁴ oocysts/chicken in an oral administration manner on the twenty-first day after the start of the breeding. At the fourth week and the fifth week after the start of breeding, the feces of the groups were sampled, and the number of oocysts per gram of the feces (OPG) was measured. The results are shown in Table 9.

TABLE 8

| Test Group Nos. | Contents of Feeds |
|---|---|
| Eleventh Group | Basal feed |
| Twelfth Group | Basal feed + Mixture 90 ppm containing pinene (4%), |
| | thymol (3%), eugenol (1%), and limonene (2%) |
| Thirteenth Group | Basal feed + Mixture 90 ppm containing pinene (4%), |
| | thymol (3%), eugenol (1%), and limonene (2%) + |
| | Mixture 0.2% of cell walls of Saccharomyces with |
| | Saccharomyce cerevisiae containing Lactobacillus |
| | (1 × 10¹⁰/g contained) |
| Fourteenth Group | Basal feed + salinomycine 50 ppm + enramycin 7 ppm |

* Pinene, thymol, eugenol, and limonene: Manufactured by TAKASAGO INTERNATIONAL CORPORATION; linomycine, and enramycin: Manufactured by Kaken Pharmaceutical Co., Ltd.,

TABLE 9

| ( ) Compared-Group | | Eleventh | | Twelfth | | Thirteenth | | Fourteenth | |
|---|---|---|---|---|---|---|---|---|---|
| Comparison | | Group | | Group | | Group | | Group | |
| Body weight | Start | 41 | (100) | 41 | (100) | 41 | (100) | 41 | (100) |
| | (g/chicken) | | | | | | | | |
| | 1 week of age | 165 | (100) | 165 | (100) | 171 | (104) | 169 | (102) |
| | (g/chicken) | | | | | | | | |
| | 2 weeks of age | 424 | (100) | 429 | (101) | 447 | (105) | 434 | (102) |
| | (g/chicken) | | | | | | | | |
| | 3 weeks of age | 855 | (100) | 863 | (101) | 891 | (104) | 897 | (105) |
| | (g/chicken) | | | | | | | | |
| | 4 weeks of age | 1383 | (100) | 1413 | (102) | 1455 | (105) | 1479 | (107) |
| | (g/chicken) | | | | | | | | |
| | 5 weeks of age | 1965 | (100) | 2057 | (105) | 2125 | (108) | 2097 | (107) |
| | (g/chicken) | | | | | | | | |
| Body weight gain | 1 week | 124 | (100) | 124 | (100) | 130 | (105) | 127 | (103) |
| | (g/chicken) | | | | | | | | |
| | 2 weeks | 259 | (100) | 264 | (102) | 276 | (107) | 266 | (103) |
| | (g/chicken) | | | | | | | | |
| | 3 weeks | 431 | (100) | 434 | (101) | 444 | (103) | 463 | (107) |
| | (g/chicken) | | | | | | | | |
| | 4 weeks | 528 | (100) | 550 | (104) | 564 | (107) | 581 | (110) |
| | (g/chicken) | | | | | | | | |
| | 5 weeks | 583 | (100) | 644 | (110) | 671 | (115) | 620 | (106) |
| | (g/chicken) | | | | | | | | |
| | Total | 1924 | (100) | 2016 | (105) | 2084 | (108) | 2057 | (107) |
| | (g/chicken) | | | | | | | | |
| Feed intake | 1 week | 199 | (100) | 187 | (94) | 193 | (97) | 198 | (99) |
| | (g/chicken) | | | | | | | | |
| | 2 weeks | 353 | (100) | 377 | (107) | 368 | (104) | 364 | (103) |
| | (g/chicken) | | | | | | | | |
| | 3 weeks | 606 | (100) | 654 | (108) | 639 | (105) | 625 | (103) |
| | (g/chicken) | | | | | | | | |
| | 4 weeks | 859 | (100) | 882 | (103) | 906 | (105) | 894 | (104) |
| | (g/chicken) | | | | | | | | |
| | 5 weeks | 1163 | (100) | 1197 | (103) | 1249 | (107) | 1148 | (99) |
| | (g/chicken) | | | | | | | | |
| | Total | 3180 | (100) | 3296 | (104) | 3354 | (105) | 3229 | (102) |
| | (g/chicken) | | | | | | | | |
| Feed conversion ratio | First week | 1.61 | (100) | 1.51 | (94) | 1.49 | (93) | 1.55 | (97) |
| | Second week | 1.36 | (100) | 1.43 | (105) | 1.33 | (98) | 1.37 | (100) |
| | Third week | 1.41 | (100) | 1.50 | (107) | 1.44 | (102) | 1.35 | (96) |
| | Fourth week | 1.63 | (100) | 1.60 | (98) | 1.61 | (98) | 1.54 | (94) |
| | Fifth week | 2.01 | (100) | 1.86 | (93) | 1.86 | (93) | 1.85 | (92) |
| | Total | 1.66 | (100) | 1.64 | (99) | 1.61 | (97) | 1.57 | (95) |
| Growth Ratio | Total (%) | 98.3 | (100) | 99.2 | (101) | 98.3 | (100) | 98.3 | (100) |
| Acervulina OPG (4, 5 | (10,000 | 23.9 | (100) | 18.8 | (79) | 18.0 | (75) | 5.5 | (23) |
| weeks in average) | oocysts/g | | | | | | | | |
| | feces) | | | | | | | | |
| Tenella OPG | (10,000 | 2.4 | (100) | 2.1 | (88) | 1.9 | (79) | 1.2 | (50) |
| (4, 5 weeks in | oocysts/g of | | | | | | | | |
| average) | feces) | | | | | | | | |
| Maxima OPG | (10,000 | 9.9 | (100) | 6.5 | (66) | 2.6 | (26) | 5.6 | (57) |
| (4, 5 weeks in | oocysts/g of | | | | | | | | |
| average) | feces) | | | | | | | | |

Referring to the results shown in Table 9, when the feed added with pinene, thymol, eugenol, and limonene was fed (to the twelfth group), the growth in the infected states of Eimeria acervulina, Eimeria tenella, and Eimeria maxima can be improved, and the excretion of OPG was suppressed. Therefore, it was determined that the infection with Eimeria could be suppressed down to a low level. In addition, due to the addition of Saccharomyces and the Lactobacillus, the growth can be further improved and the excretion of OPG tended to be lowered. As a result, it was determined that a more excellent anticoccidial effect could be obtained (the thirteenth group).

### Example 4

5 groups (20 chickens/group) of tested chickens (type of the chicken: chunky, neonatal chick, male) were prepared.

The fifteenth to eighteenth groups of tested chickens are bred for 21 days by freely feeding the feeds (Table 10) obtained by adding the ingredients to the basal feed (100 mass %) shown in Table 1. The basal feed used in the test is mixed with 30% of rye. Therefore, proliferation of clostridium perfringens in an intestinal canal is facilitated, so that the growth of the neonatal chickens is suppressed. At the first, second, and third weeks after the start of breeding, the number of clostridium perfringens was measured by using a cloacal swab. The results are shown in Table 11.

TABLE 10

| Test Group Nos. | Contents of Feeds |
|---|---|
| Fifteenth Group | Basal feed |
| Sixteenth Group | Basal feed + enramycin 7 ppm |
| Seventeenth | Basal feed + Mixture 90 ppm containing pinene (4%), |
| Group | thymol (3%), eugenol (1%), and limonene (2%) |
| Eighteenth Group | Basal feed + Mixture 90 ppm containing pinene (4%), |
| | thymol (3%), eugenol (1%), and limonene (2%) + |
| | Mixture 0.2% of cell wall of Saccharomyces with |
| | Saccharomyces cerevisiae containing Lactobacillus |
| | (1 × 10¹⁰/g contained) |

* Enramycin: Manufactured by BASF; pinene, thymol, eugenol, and limonene: Manufactured by TAKASAGO INTERNATIONAL CORPORATION

TABLE 11

| ( ) Compared- | | | | | |
|---|---|---|---|---|---|
| Group | | Fifteenth | Sixteenth | Seventeenth | Eighteenth |
| Comparison | | Group | Group | Group | Group |
| Body | Start | 41 (100) ± 2 | 41 (100) ± 2 | 41 (100) ± 2 | 41 (100) ± 2 |
| weight | (g/chicken) | | | | |
| | 1 week of age | 120 (100) ± 17 | 136 (114) ± 12 | 137 (114) ± 16 | 127 (106) ± 16 |
| | (g/chicken) | | | | |
| | 2 weeks of age | 286 (100) ± 62 | 356 (124) ± 37 | 317 (111) ± 49 | 304 (106) ± 45 |
| | (g/chicken} | | | | |
| | 3 weeks of age | 541 (100) ± 99 | 700 (130) ± 70 | 581 (107) ± 98 | 617 (114) ± 92 |
| | (g/chicken) | | | | |
| Body weight | 1 week | 79 (100) | 96 (121) | 96 (121) | 86 (109) |
| gain | (g/chicken) | | | | |
| | 2 weeks | 167 (100) | 220 (132) | 180 (108) | 177 (106) |
| | (g/chicken | | | | |
| | 3 weeks | 254 (100) | 345 (135) | 264 (104) | 313 (123) |
| | (g/chicken | | | | |
| | Total | 500 (100) | 660 (132) | 540 (108) | 577 (115) |
| | (g/chicken) | | | | |
| Feed intake | 1 week | 110 (100) | 118 (107) | 125 (114) | 115 (105) |
| | (g/chicken) | | | | |
| | 2 weeks | 230 (100) | 323 (140) | 313 (136) | 275 (120) |
| | (g/chicken | | | | |
| | 3 weeks | 412 (100) | 510 (124) | 433 (105) | 488 (119) |
| | (g/chicken | | | | |
| | Total | 752 (100) | 950 (126) | 871 (116) | 878 (117) |
| | (g/chicken) | | | | |
| Feed | First week | 1.39 (100) | 1.23 (89) | 1.30 (94) | 1.33 (96) |
| conversion | | | | | |
| ratio | | | | | |
| | Second week | 1.38 (100) | 1.47 (106) | 1.74 (126) | 1.55 (112) |
| | Third week | 1.62 (100) | 1.48 (91) | 1.64 (101) | 1.56 (96) |
| | Total | 1.50 (100) | 1.44 (96) | 1.61 (107) | 1.52 (101) |
| Number of | First week | 6.0 (100) | 2.0 (34) | 4.1 (69) | 4.7 (79) |
| clostridium | (LogCFU/g) | | | | |
| perfringens | | | | | |
| | Second week | 4.3 (100) | 2.0 (47) | 5.0 (117) | 3.3 (78) |
| | (LogCFU/g) | | | | |
| | Third week | 4.8 (100) | 2.0 (41) | 5.1 (105) | 3.0 (62) |
| | (LogCFU/g) | | | | |
| | Total | 5.0 (100) | 2.0 (40) | 4.7 (94) | 3.7 (73) |

Referring to the results shown in Table 11, the feed added with pinene, thymol, eugenol and limonene (the seventeenth group) suppressed the initial proliferation of clostridium and improved weight gain under the experimental condition that feeding of the basal feed added with a higher amount of rye facilitated the proliferation of the clostridium and thus enlarged the influence of the clostridium to growth performance. Therefore, the influence of the clostridium infection can be reduced. In addition, due to the addition of Saccharomyces and Lactobacillus, the proliferation of the clostridium can be further suppressed, and the weight increment ratio was increased. As a result, it was determined that it was possible to obtain an excellent anticlostridial effect (the eighteenth group).

What is claimed is:

1. A prophylactic and/or therapeutic feed for coccidiosis, comprising pinene, thymol, eugenol, and limonene.

2. A prophylactic and/or therapeutic feed for coccidiosis, comprising nutmeg, thyme, clove, and citrus.

3. The feed according to claim 1 or 2, further comprising Saccharomyces and Lactobacillus

4. An anticoccidial agent, comprising pinene, thymol, eugenol, and limonene as active ingredients.

5. An anticoccidial agent, comprising nutmeg, thyme, clove, and citrus.

6. The anticoccidial agent according to claim 4 or 5, further comprising Saccharomyces and Lactobacillus.

7. A prophylactic and/or therapeutic feed for clostridial disease, comprising pinene, thymol, eugenol, and limonene.

8. A prophylactic and/or therapeutic feed for clostridial disease, comprising nutmeg, thyme, clove, and citrus.

9. The feed according to claim 7 or 8, further comprising Saccharomyces and Lactobacillus.

10. An anticlostridial agent, comprising pinene, thymol, eugenol, and limonene as active ingredients.

11. An anticlostridial agent, comprising nutmeg, thyme, clove, and citrus.

12. The anticlostridial agent according to claim 10 or 11, further comprising Saccharomyces and Lactobacillus.
